(19) United States
(12) Patent Application Publication
(10) Pub. No.: US 2022/0080220 A1
(43) Pub. Date: Mar. 17, 2022

(54) OPHTHALMIC ILLUMINATION DEVICE

(71) Applicant: ContactRx LLC, Pasadena, CA (US)

(72) Inventors: Nicholas E. SCIANMARELLO, Monrovia, CA (US); Mark S. HUMAYUN, Glendale, CA (US); Yu-Chong TAI, Pasadena, CA (US); Andrew URAZAKI, Las Vegas, NV (US)

(21) Appl. No.: 17/470,160

(22) Filed: Sep. 9, 2021

Related U.S. Application Data
(60) Provisional application No. 63/077,287, filed on Sep. 11, 2020.

(51) Int. Cl. A61N 5/06; A61F 9/007

(52) U.S. Cl. CPC A61N 5/0613; A61F 9/007; A61N 2005/0662; A61N 2005/063; A61N 2005/0665; A61N 2005/0651

(57) ABSTRACT

An ophthalmic device for illuminating an interior region of a patient's eye includes a polymeric, at least partially transparent body having a first surface contoured to substantially conform to an ocular surface and a second surface opposed to the first surface; a light source; and an optical element for directing light through the first surface such that, with the first surface in contact with a corneal surface of the patient's eye, a portion of interior region of the patient's eye illuminated by the light source is visible through the first and second surfaces. In certain embodiments, the light source is sufficient to illuminate the retina so that an ophthalmic surgeon may view the eye during an evaluation or surgical procedure without the need for invasive probes, cameras, or other equipment.

### CROSS-REFERENCE TO RELATED APPLICATION

This application claims priority to and the benefit of, and incorporates herein by reference in its entirety, U.S. Ser. No. 63/077,287, filed on Sep. 11, 2020.

### FIELD OF THE INVENTION

In various embodiments, the present invention relates generally to an ophthalmic illumination device for placement on the corneal and/or scleral surface of the eye. The lens illuminates and/or provides a phototherapeutic treatment to the tissues of the eye. Alternatively, the lens provides a non-invasive lighting function to illuminate the interior physiology of the eye for evaluation and/or during surgical procedures.

### BACKGROUND

Many ophthalmological conditions, such as diabetic retinopathy, age-related macular degeneration, and retinopathy of prematurity arise from aberrant angiogenesis, driven in part by expression of vascular endothelial growth factor (VEGF) in response to cellular oxygen deprivation. The oxygen tension within the retina of the eye is of primary concern in these diseases and is a function of supply (oxygen diffusion from the choroid and retinal capillaries) and demand (primarily from photoreceptors and nerve cells). The retina is a multilayered structure composed of various photoreceptor and nerve cells sandwiched between the retinal and choroidal blood supply. Consequently, oxygen delivery to the cells of the retina occurs by oxygen diffusion from either the retinal vasculature or the choroid. This puts an upper limit on the amount of oxygen that can be delivered to the cells within the retina. It has been shown that the metabolic demands of photoreceptors (primarily rods) are inversely proportional to the amount of light they are exposed to. Consequently, metabolic demands are significantly higher in the dark. The increase in rod metabolism during dark adaptation can lead to hypoxia within the retina as demand outstrips diffusional supply. In patients with compromised retinal circulation, such as diabetics, the elderly, or premature babies, the effect is amplified. This is known as rod-driven hypoxia and is becoming understood as a driver for pathogenesis.

Ultimately, treatment of ophthalmological pathologies with hypoxic etiology requires either reversing the oxygen deficiency or interrupting the resulting angiogenic cascade. Several approaches have been developed along these lines. The most clinically significant approach today is the administration of VEGF antagonists into the eye to block the signaling of angiogenesis. This can reduce the ingrowth of new blood vessels onto the retina, which helps mitigate vision loss; it does not, however, treat the underlying cause of the disease—hypoxia.

Other approaches have enhanced oxygen delivery to the retina by means of implants, which locally increase oxygen tension around the retina to increase diffusional supply. Both passive devices, which shunt atmospheric oxygen from the surface of the eye through to the retina, and active devices, which generate oxygen through electrolysis, have been developed and demonstrated. The clinical efficacy of these approaches has not yet been established. Neither of these approaches, however, addresses the fact that dark adaptation drives hypoxia through increased rod metabolism. It has been proposed that, by stimulating the rod cells with low levels of light, it may be possible to reduce their metabolic demand for oxygen and thereby reduce or eliminate hypoxia.

As ophthalmic disease cases rise along with an aging global population, there is a growing need for a non-invasive, low-cost option allowing ophthalmic surgeons to visualize and examine a patient's eye. Current methods used during surgical procedures typically require high-cost capital equipment, invasive light probes that must be inserted through a surgical incision, or external light sources that crowd the already busy surgical area without directly illuminating the eye. Therefore, new ophthalmic illumination device configurations capable of addressing these escalating and difficult-to-reconcile requirements for surgical use and therapeutic effect are constantly being sought.

### SUMMARY

In various embodiments, the present invention relates generally to an ophthalmic illumination device for placement on the corneal and/or scleral surface of the eye. The device illuminates and/or provides a phototherapeutic treatment to the tissues of the eye. In certain embodiments, the light source is sufficient to illuminate a portion of the retina so that an ophthalmic surgeon may view the eye during an evaluation or surgical procedure without the need for invasive probes, cameras, or other equipment. In other embodiments, the lens is similar in device size to a contact lens and can be worn by a user for extended periods of time to provide a phototherapeutic effect.

Accordingly, in one aspect, the invention pertains to an ophthalmic device for illuminating an interior region of a patient's eye. In various embodiments, the device comprises a polymeric, at least partially transparent body having a first surface contoured to substantially conform to an ocular surface and a second surface opposed to the first surface; a light source; and an optical element for directing light through the first surface such that, with the first surface in contact with a corneal surface of the patient's eye, a portion of interior region of the patient's eye illuminated by the light source is visible through the first and second surfaces.

In some embodiments, the light source is centered on a central axis of the optical element, while in other embodiments, the light source is offset from the central axis. The body may or may not fully encapsulate the light source, which may be a light-emitting diode. In various embodiments, the device includes one or more secondary light sources having a wavelength or intensity different from the wavelength or intensity of the primary light source. The device may include a power coil for receiving power by induction and supplying power to the light source.

In some embodiments, the device further comprises a controller for controlling operation of the light source. The device may include at least one focusing element to alter the light directed to the patient's eye. The focusing element(s) may be adjustable to alter at least one of position, refraction, reflection, optical power, or backscatter of the light. For example, the focusing element(s) may be one or more lenses.

The device may include at least one opening through the outer shell to permit adequate oxygenation to the corneal surface. The device may have a raised handle projecting from the body away from the first surface. For example, the handle may be disposed above the light source, thereby minimizing light backscatter. The device may include at least one light-absorbing layer to minimize light back-reflection and/or at least one light-reflecting layer to increase light transmittance to the patient's eye. Some or all surfaces of the body may be coated to prevent external light from entering and to reduce internal light scatter. The device may include blue light-absorbing chromophores. The ocular surface on which the device is configured for placement may be the cornea or the sclera.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean ±10%, and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 illustrates the generalized anatomy of the eye onto which the non-invasive illumination device may be placed.

FIG. 2 schematically illustrates prior-art surgical options for ocular illumination used in ophthalmic surgery.

FIG. 3A schematically illustrates the operation of an ophthalmic surgical illumination device illuminating the interior of the eye in accordance with embodiments of the present disclosure.

FIGS. 3B-3D are schematic sectional views of different embodiments of an ophthalmic surgical illumination device in accordance herewith.

FIG. 4 illustrates various views of an ophthalmic surgical illumination device as illustrated in FIGS. 3B-3D.

FIG. 5 is a plan view of an ophthalmic surgical illumination device and the blood vessels of the retina that are clearly illuminated by the device.

FIG. 6 illustrates an example of a mold to manufacture a surgical illumination device according to embodiments of the present disclosure.

FIGS. 7A and 7B are schematic sectional views of a wearable illumination device, according to embodiments of the present disclosure.

FIG. 8 illustrates various views of wearable illumination device as illustrated in FIGS. 7A and 7B.

### DETAILED DESCRIPTION

Embodiments of the present disclosure are directed to an ophthalmic illumination device for placement on the corneal and/or scleral surface of the eye. The various embodiments described herein overcome the main challenge of external endoillumination by a surgical microscope or other external light source that is difficult to position and has various light artifacts caused by unwanted reflection and light scatter from ocular structures, i.e., the cornea, lens, and vitreous fluid. Embodiments of the invention also overcome challenges associated with typical insertable endoilluminator devices, e.g., the need for an additional sclerotomy for insertion of a chandelier endoilluminator, an elevated risk of phototoxicity due to direct light source placement in the vitreous cavity, shadowing due to positioning of the light source with respect to other surgical devices, and potential contact with sensitive tissue such as the retina during insertion. Comparatively, embodiments described herein invention provides satisfactory illumination while maintaining the principal advantage of enabling surgeons to conduct bimanual surgery without the need to continuously hold or reposition an endoilluminator.

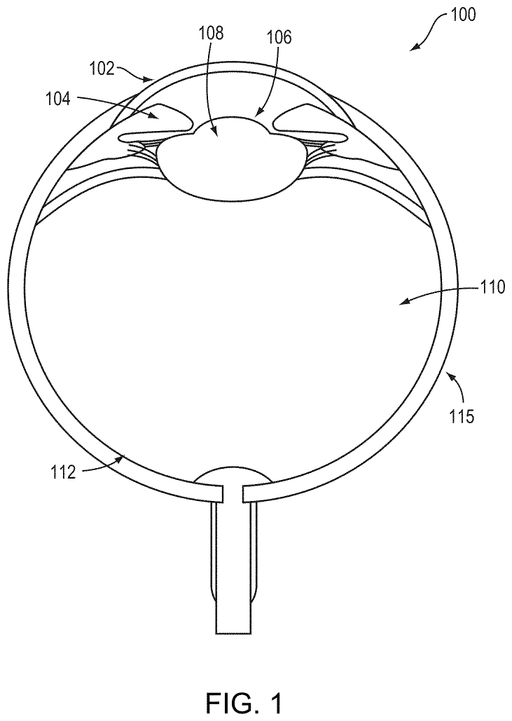

FIG. 1 illustrates the anatomy of the eye onto which the non-invasive illumination device may be placed. The eye 100 includes the cornea 102, iris 104, pupil 106, lens 108, vitreous body 110, retina 112, and sclera 115. Light enters the eye 100 through the clear, dome-shaped cornea 102. The iris 104 is the colored part of the eye and controls the amount of light entering the eye. The pupil 106 is the central opening of the iris 104 through which light passes and enters the lens 108, which focuses light onto the retina 112 as it travels through the vitreous body 110. Light does not enter other than through the cornea as the sclera 115, the white external part of the eye, covers the remaining entire surface of the eye 100.

Devices as described herein illuminate anatomical areas of the eye 100, specifically areas of the retina 112. In certain embodiments, the light source is sufficient to illuminate the retina 112 so that an ophthalmic surgeon may view the eye during an evaluation or surgical procedure without the need for invasive lighting systems, probes, cameras, or other equipment. Devices in accordance with the invention may be similar in size to a contact lens and can be worn by a user for extended periods of time.

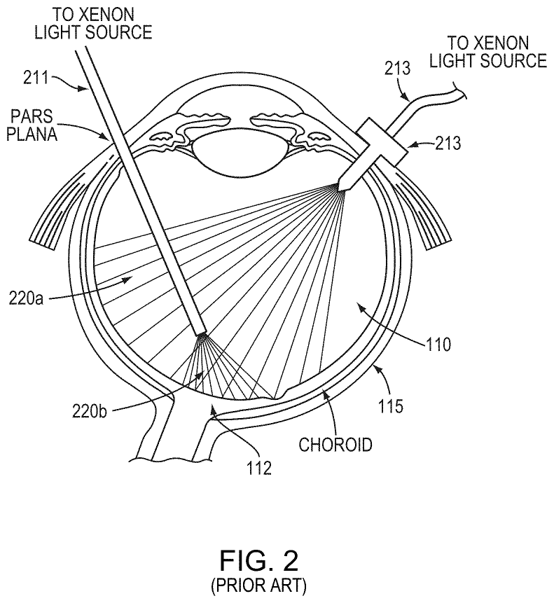

In contrast to the present invention and as shown in FIG. 2, conventional insertable endoilluminator tools 211, 212 used during vitreo-retinal surgeries are inserted through a sclerotomy, or incision in the sclera 115 usually at the pars plana, and pass into the vitreous body 110. Only one sclerotomy is made in each of the four quadrants of the eye, so the number of tools insertable into the eye is limited. Each sclerotomy has a potential risk of vitreous leakage, hypotony, infection, and dislocation of the trocar or light during the surgical procedure. The endoilluminator tool 211 includes a rigid probe that is inserted into the vitreous body 110 and can be manipulated to illuminate specific areas of the sclera 115 by generating a cone of light 220b. While moving the endoilluminator tool 211, one of the surgeon's hands is occupied. The endoilluminator tool 212 is a fixed chandelier type that hangs in a fixed trocar 213 and provides a wider cone of light 220a. As the chandelier endoilluminator 212 is fixed, the surgeon is free to use both hands for other tasks during the surgery. However, precisely because it is fixed, the chandelier endoilluminator tool 212 may cast a shadow depending on the relative placement of other instruments during surgery. As a result, certain surgeries require a second chandelier endoilluminator be inserted in a different quadrant of the eye to mitigate the shadow.

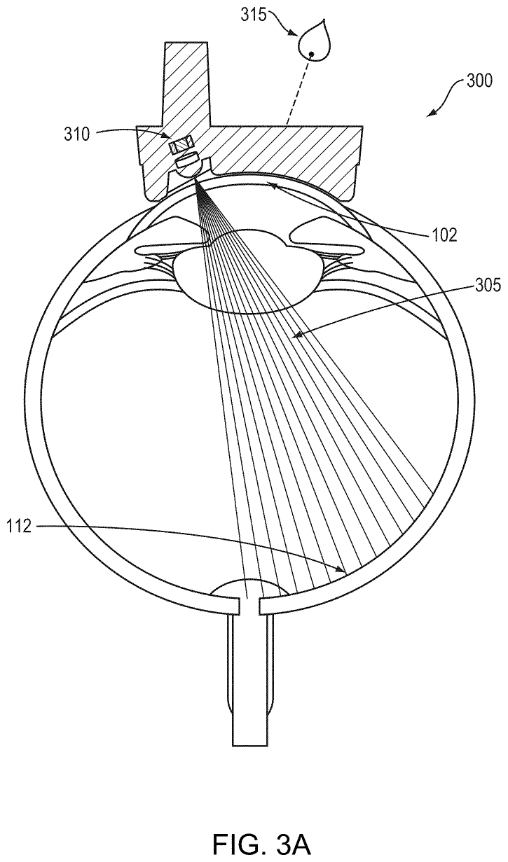

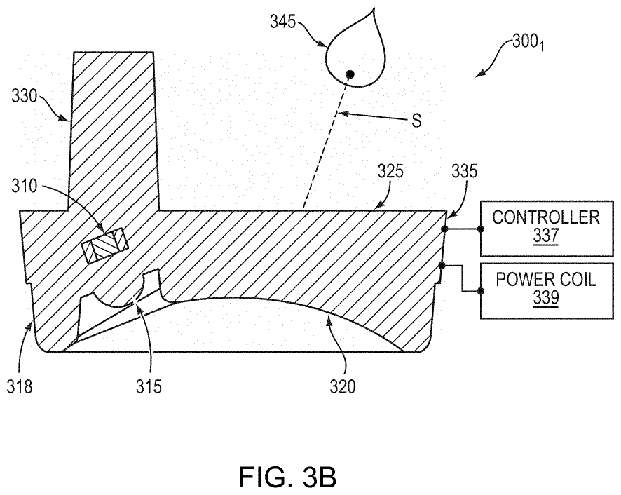

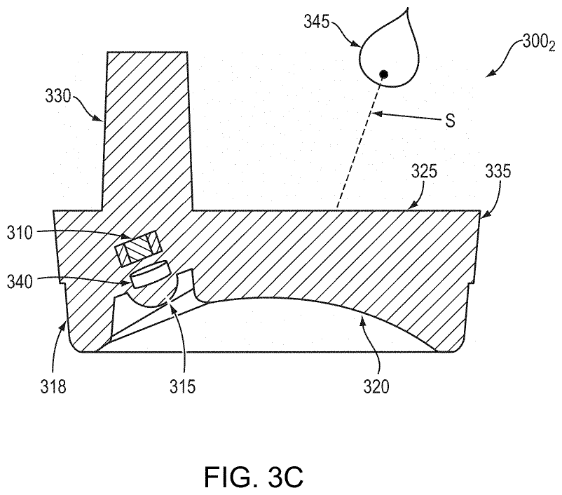

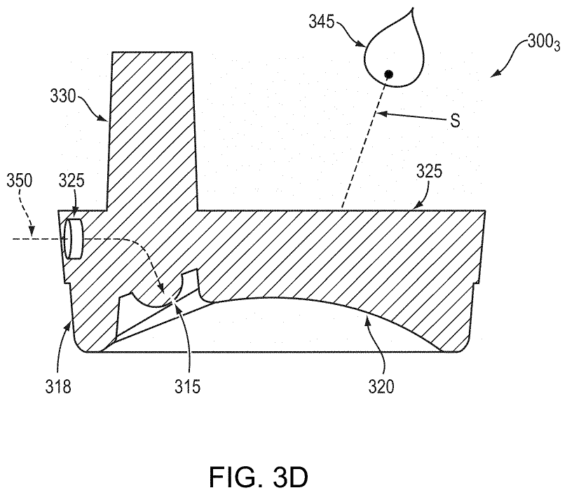

FIGS. 3A and 3B illustrate one embodiment of the surgical illumination device, indicated as 300₁, and the cone of light 305 that it produces to illuminate the retina 112. In use, the surgical illumination device 300₁ is placed on the cornea 102, and a light source 310 is activated to illuminate the interior of the eye. The device 300₁ may be freely moved around to alter both the path of the light cone 305 produced by the light source 310 (to illuminate different areas of the retina) and the viewing window visible to the surgeon's eye 345 as s/he peers through the device 300₁. Depending on the procedure being performed, the surgeon may topically apply gel, wetting agent, anesthesia, or irrigation fluid to the surface of the cornea 102. The surgical illumination device 300₁ may be placed over any of these fluids. As shown in FIG. 3B, in various embodiments, the emission components of the surgical illumination device 300₁ include the light source 310 and an optical element 315. Typically the light source 310 is a light-emitting diode (LED) and the optical element 315 is a lens that shapes the LED output so that the light will illuminate a substantial portion (e.g., 25% to 75% of the visible area) of the retina 112 with the device 300₁ in place on the patient's cornea 102. The focal center of light and curvature of the optical element 315 determine the focus and spread of the cone of light that travels through the eye anatomy. The light source 310 may be any suitable light-producing device including radioluminescent, chemiluminescent, and/or electroluminescent devices. It may be centered on the central axis of the optical element 315 or may instead be offset therefrom.

The emission components are retained within the body of the device 300₁, which may be an optically transparent polymer as described below. The device body 318 is shaped to include a posterior surface 320, which has a contour shaped to conform to the cornea 102; an anterior surface 325, and a handle portion 330. The optical element 315 may be molded directly into the body 318 or affixed within a molded recess. In embodiments where the optical element 315 is molded directly into the body 318, the posterior surface 320 is continuous and does not include a recess. A flanged peripheral edge 335 may include a battery, an electronic controller 337, and/or a wireless power transfer coil 339 to power the light source 310 and facilitate wireless communication with the device 300₁. By including these components around the periphery of the device, they do not interfere with the surgeon's line of sight S. In embodiments not including a controller, the battery may instead be integrated with the light source 310. In still other embodiments, the light source 310 is powered by a fine wire entering the device 300.

The light source 310 and/or the handle 330 may be backed with a light-reflective (e.g., metallic) or light-absorbing (e.g., carbon black) material to prevent back-reflections and help focus the light onto the retina 112 by minimizing unwanted light scatter from other surfaces of the surgical illumination device 300₁. In this embodiment, the light source 310 is located beneath the handle 330 to further limit back-reflection through the anterior surface 325. The light illuminates the retina 112 or other anatomical landmark and is visible through the anterior surface 325. The posterior surface 320, as noted, has a curvature approximating the average curvature of the human cornea—i.e., a radius of curvature 7.8 mm with variation less than 0.3 mm. It is of course possible to more closely match the corneal curvature of a particular patient by providing a selection of devices 300 with posterior surfaces 320 having different curvature radii, e.g., 7.6 mm, 7.7 mm, 7.8 mm, 7.9 mm, and 8 mm. The anterior surface 325 may also have a curvature to provide a desired degree of magnification (i.e., a more concave surface provides higher magnification). In other embodiments, the posterior surface 320 may be shaped to conform to the sclera.

All exposed surfaces of the device 300 (i.e., other than the surface 320 and, in some embodiments, the surface 325) may be coated (e.g., with an antireflective or non-light permeating coating) to prevent external light from entering the device and to minimize light scatter therewithin.

In an alternative embodiment 300₂ illustrated in FIG. 3C, a secondary optical element 340 is disposed between the light source 310 and the primary optical element 315. The secondary optical element 340 may comprise or consist of one or more of a light collimator, a diffraction grating, a bandpass filter, blue light-chromophores, tinting, a contrast-improving filter, a color filter, one or more macro-filters, one or more lenses, reflectors, prisms, or focusing elements, a fiber optic, or an optical gas pocket—or other structure known to control the properties and direction of the light. In various embodiments, the position of the focusing element 340 is adjustable to alter at least one of position, refraction, optical power, or backscatter of the light directed through the posterior surface 320. Alternatively, the light source 310 may be embedded or partially encapsulated within a secondary optical element 340 such as a fiber optic or reflective metallic tube to focus the light and minimize diffraction of light entering the primary optical element 315, thereby minimizing backscatter and removing other optical artifacts such as halos, glares, and unwanted reflection (which may blur or wash out the image seem by the surgeon). Furthermore, a light-absorbing pigment may be incorporated into the body material or parts of the anterior surface 325 and handle 330 through which light should not pass.

In yet another embodiment 300₃ illustrated in FIG. 3D, the light source is a fiber-optic system used in a commercially available endoillumination lighting system, e.g., a system manufactured by Alcon Laboratories Inc., Bausch+Lomb, Dutch Ophthalmic Research Center (DORC), etc. The optical fiber 350 from the endoillumination lighting system is inserted into a fiber-optic port 355 and the entering light is guided to the optical element 315. The fiber-optic port 355 may be a specific size (e.g., 25 gauge, 27 gauge, 29 gauge, etc.) or may define a tapered channel that can admit and retain optical fibers of any size.

In further alternative embodiments, the surgical illumination device 300 may have two or more light sources 310 and paired optical elements 315 located in different areas to allow for simultaneous illumination, to generate more light, or with switching features to turn one or more light sources 310 with differing wavelength or intensity on and off. The switching feature may be realized in a controller 337 addressable wirelessly or as a manipulatable switch (e.g., on the handle 330). In some embodiments, the surgical illumination device 300 includes one or more focusing elements to alter the light directed into the eye.

As noted, the surgical illumination device 300 may include a power coil 339 to receive power wirelessly, by electromagnetic induction, from a charging coil—either to charge an internal battery or to substitute entirely for an internal power supply. An electrically powered device may further include one or more sensors (e.g., a light sensor to ensure that the emitted light is below a phototoxicity threshold, a tear film analyzer, retinal electrocardiogram sensor, etc.), and in such embodiments, the controller 337 may include a telemetry unit for data exchange with an external telemetry unit and/or a built-in memory unit (which may be part of the controller 337).

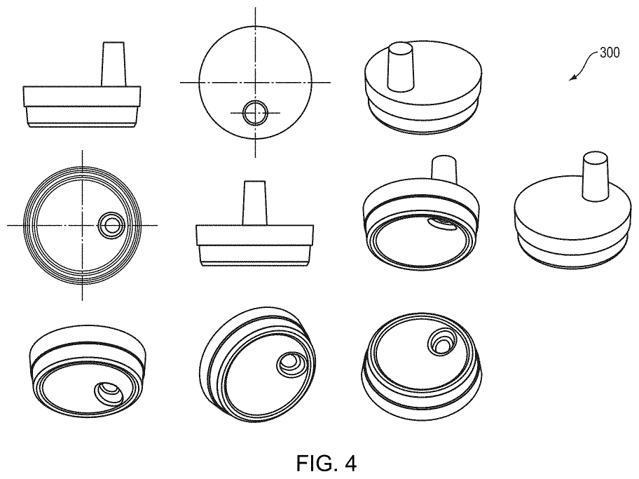

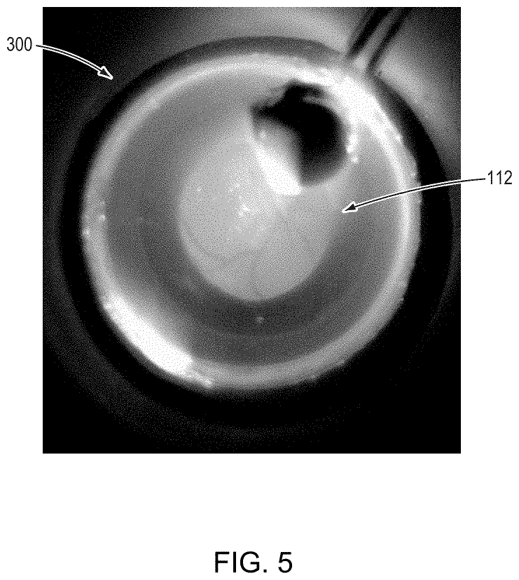

FIG. 4 illustrates various views of the surgical illumination device 300. The top orthogonal views show how easily the handle may be manipulated with forceps during surgery to either move or rotate the surgical illumination device 300 to achieve the appropriate illumination and angle. FIG. 5 shows the device 300 in use, illustrating how the blood vessels of the retina 112 are illuminated by the device and clearly visible through the anterior surface. Clear visualization of the retina is required for successful vitrectomy surgery.

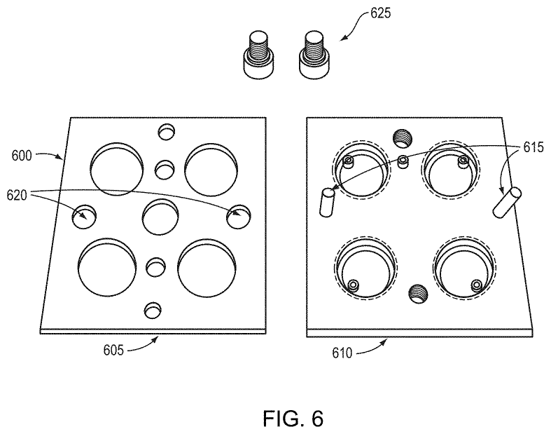

The surgical illumination device 300 may be manufactured using a two-piece mold 600 as shown in FIG. 6. The mold 600 includes complementary elements 605, 610, and is ideally made of a hard metal, such as stainless steel, that can be polished to an optical finish. The surgical illumination device is cast in the mold using an optically transparent polymer such as medical-grade silicone (e.g., the NUSIL MED series silicone supplied by Avantor) in multiple parts to allow embedding or encapsulation of key components such as the light source 310 and optical element 315. For proper alignment, the mold 600 may include a pair of alignment pins 615 on the mold piece 610 that pass through complementary apertures 620 on the mold piece 605. Set screws 625 may be used to keep the mold pieces 605, 610 tightly clamped during curing.

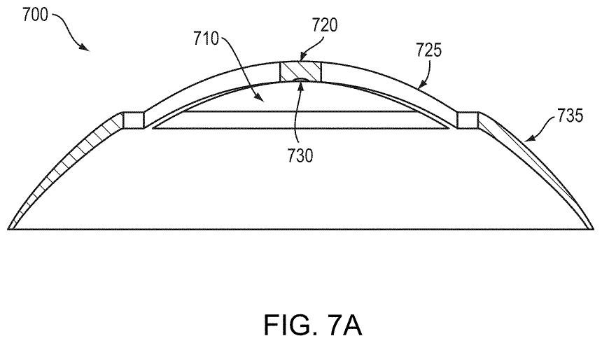

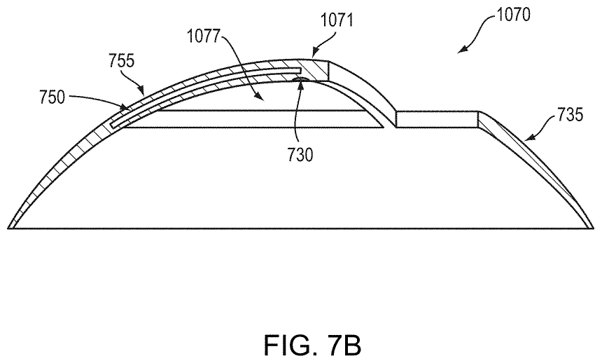

FIG. 7A illustrates a wearable illumination device 700 according to embodiments of the present disclosure. The device 700 is similar in size to a contact or scleral lens and can be worn by a user for extended periods of time to provide a phototherapeutic effect. Specifically, light is used to modulate retinal metabolism and oxygenation, representing a potential preventative treatment for diabetic retinopathy by mitigating hypoxia and subsequent VEGF expression during daily sleep cycles. The wavelength of the light should overlap with the maximum absorbance of rod cells/rhodopsin while being far from the maximal absorbance of blue or green cones, thereby maximizing the efficiency of the phototherapy while minimizing the visual side effects of continuous phototherapy (such as sleep disruption). As many light sources (such as radioluminescent and chemiluminescent light source) have waning luminescence that depends on external ionizing radiation and chemical reactions, LED light sources may be preferred. The LED and associated circuit can be selected to emit certain wavelengths (e.g., 400 nm and 600 nm) at a set irradiance onto the retina (10⁹ to 10¹¹ photons/s-cm²) to produce a phototherapeutic effect for mitigating ocular hypoxia. The phototherapeutic effect may additionally or alternatively have photobiomodulation effects according to the light wavelength, irradiance, and intensity selected for treatment of various ocular diseases.

An open volume 710 over the cornea allows oxygen transmission for long-term wear such as six to ten hours during daily sleep. The light source 720 is placed centrally above the open space 710 and held in place by two or more spokes 725 that arc across the open space 710. An optical element 730 is positioned posterior to the light source 720, allowing light from the light source 720 to be transmitted through the optical element 730 to the cornea. In certain embodiments utilizing an LED or other electronically powered light source 720, the peripheral circumferential edge 735 may house a battery, electronic controller, or wireless power-transfer coil as described above.

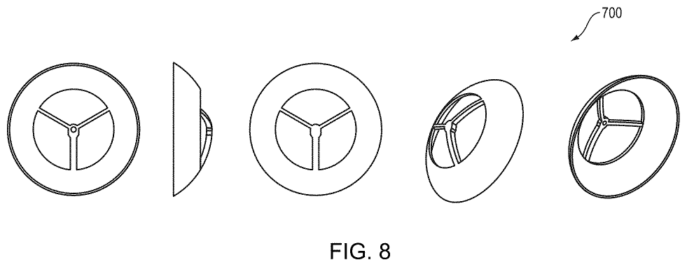

FIG. 7B illustrates a cross-sectional cut view of the wearable illumination device 700, according to the embodiments of the present disclosure. In this embodiment, an optical fiber 750 is embedded within the peripheral edge 755, or the latter is constructed of two materials with different refraction indices in order to conduct light as a waveguide. The optical fiber 750 or waveguide allows light to propagate along the spoke 755, allowing for greater illumination of the retina. FIG. 8 illustrates various views of the wearable illumination device 700.

The controller 337 may be provided as either software, hardware, or some combination thereof. Typically the controller includes a microcontroller with associated memory unit for storing programs and/or data relating to operation of the devices described above. The memory may include random access memory (RAM), read only memory (ROM), and/or FLASH memory. Code for operating the microcontroller may be written in assembler or other native microcontroller language, or in a high-level language such as JAVA, PYTHON, C, C++, various scripting languages, and/or HTML.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An ophthalmic device for illuminating an interior region of a patient's eye, the device comprising:
a polymeric, at least partially transparent body having a first surface contoured to substantially conform to an ocular surface and a second surface opposed to the first surface;
a light source; and
an optical element for directing light through the first surface such that, with the first surface in contact with a corneal surface of the patient's eye, a portion of interior region of the patient's eye illuminated by the light source is visible through the first and second surfaces.

2. The device of claim 1, wherein the light source is centered on a central axis of the optical element.

3. The device of claim 1, wherein the light source is offset from a central axis of the optical element.

4. The device of claim 1, wherein the body fully encapsulates the light source.

5. The device of claim 1, wherein the light source is an LED.

6. The device of claim 1, further comprising one or more secondary light sources having a wavelength or intensity different from a wavelength or intensity of the light source.

7. The device of claim 1, further comprising a power coil for receiving power by induction and supplying power to the light source.

8. The device of claim 1, further comprising a controller for controlling operation of the light source.

9. The device of claim 1, further comprising at least one focusing element to alter the light directed to the patient's eye.

10. The device of claim 9, wherein the at least one focusing element is adjustable to alter at least one of position, refraction, reflection, optical power, or backscatter of the light.

11. The device of claim 9, wherein the at least one focusing element is a lens.

12. The device of claim 1, further comprising at least one opening through the outer shell to permit adequate oxygenation to the corneal surface.

13. The device of claim 1, further comprising a raised handle projecting from the body away from the first surface.

14. The device of claim 13, wherein the handle is disposed above the light source, thereby minimizing light backscatter.

15. The device of claim 1, further comprising at least one light-absorbing layer to minimize light back-reflection.

16. The device of claim 1, further comprising at least one light-reflecting layer to increase light transmittance to the patient's eye.

17. The device of claim 1, wherein surfaces of the body are coated to prevent external light from entering and to reduce internal light scatter.

18. The device of claim 1, wherein the ocular surface is the cornea.

19. The device of claim 1, wherein the ocular surface is the sclera.

20. The device of claim 1, further comprising blue light-absorbing chromophores.
